(19) United States
(12) Patent Application Publication
(10) Pub. No.: US 2020/0283744 A1
(43) Pub. Date: Sep. 10, 2020

(54) APPLICATION OF CRISPR/CAS12A GENE EDITING SYSTEM IN GENE EDITING OF PHYSCOMITRELLA PATENS

(71) Applicant: KUNMING INSTITUTE OF BOTANY, CHINESE ACADEMY OF SCIENCES, YUNNAN (CN)

(72) Inventors: Li Liu, Yunnan (CN); Xiaojun Pu, Yunnan (CN); Lina Liu, Yunnan (CN); Ping Li, Yunnan (CN); Hong Yang, Yunnan (CN)

(21) Appl. No.: 16/508,712

(22) Filed: Jul. 11, 2019

(30) Foreign Application Priority Data
Mar. 4, 2019 (CN) 201910159312.X

(51) Int. Cl. C12N 9/22; C12N 15/11; C12N 15/82

(52) U.S. Cl. CPC C12N 9/22; C12N 2310/20; C12N 15/8202; C12N 15/11

(57) ABSTRACT

Some embodiments of the disclosure provide an application method of a CRISPR/Cas12a gene editing system in the gene editing of Physcomitrella patens (P. patens). According to an embodiment, the application method includes the following steps: 1) constructing a Cas12a protease expression vector by ligating a Cas12a-protease-encoding nucleotide sequence with nuclear localization signals at both ends to a plasmid pAct-Cas9 and initiating expression by a pActin promoter; 2) constructing a gRNA expression vector by ligating a gRNA to a plasmid pU6-sgRNA, and initiating expression by a PpU6 promoter; and 3) transforming P. patens by using the Cas12a protease expression vector, the gRNA expression vector, and the plasmid for resistance expression obtain a mutant plant through screening for resistance.

### CROSS REFERENCE TO RELATED APPLICATIONS

This application claims priority to Chinese application number 20191015931-2.X filed on Mar. 4, 2019, the disclosure of which is incorporated by reference herein in its entirety.

### FIELD OF THE DISCLOSURE

The disclosure relates generally to the field of genetic engineering. More specifically, the disclosure relates to the field of the application of a CRISPR/Cas12a gene editing system in the gene editing of Physcomitrellapatens (P. patens).

### BACKGROUND

Physcomitrella patens is categorized into the genus Physcomitrella of the family Funariaceae, and is distributed in Europe, Asia, Africa and Oceania, and distribution of it is found in the area of Zhangjiajie, Hunan Province, China.

P. patens requires simple nutrients for growth and is easy in cultivation; its gametophyte is dominant in the life history, and thus the phenotype of its mutant can be directly studied. High-frequency homologous recombination easily occurs between the nuclear genome of P. patens and a foreign DNA having a fragment homologous thereto, such that it is possible to make accurate gene disruption and gene knockout, providing good materials for studying of gene functions. P. patens has many similar characteristics to higher terrestrial plants, and some of the characteristics of P. patens make it easier to conduct molecular biology study on it than other plants. P. patens has become a model organism for molecular biology study of plants in foreign countries.

Knockout is an exogenous DNA introduction technology in which a DNA fragment containing a certain known sequence is homologously recombined with a gene in a genome of a recipient cell. The gene has a sequence identical or similar to the DNA fragment, and incorporating into the genome of the recipient cell and then expressing. It alters the genetic gene of an organism on a known sequence with an unknown function, and thus disables a specific gene function, so that some functions are blocked and the organism can be further affected, thereby inferring the biological function of the gene.

Currently, the method for conducting gene knockout of P. patens is mainly to use homologous recombination, the efficiency of conducting double-gene knockout simultaneously is less than 4% by using the method. However, a multi-gene-knockout requires different combinations of resistance, limiting the availability of multiple knockout mutants.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

Some embodiments of the disclosure provide an application of a CRISPR/Cas12a gene editing system in the gene editing of P. patens.

In some embodiments an application of a CRISPR/Cas12a gene editing system in the gene editing of P. patens includes the following steps: 1) ligating a Cas12a-protease-encoding nucleotide sequence with nuclear localization signals at both ends to a plasmid pAct-Cas9, and initiating expression by a pActin promoter to obtain a Cas12a protease expression vector; 2) ligating a gRNA to a plasmid pU6-sgRNA, and initiating expression by a PpU6 promoter to obtain a gRNA expression vector; and 3) transforming P. patens by using the Cas12a protease expression vector of step 1), the gRNA expression vector of step 2), and a plasmid for screening of resistance expression to obtain a mutant plant through screening for resistance.

In other embodiments, there is no limitation on the temporal order of step 1) and step 2). The Cas12a-protease-encoding nucleotide sequence with nuclear localization signals at both ends is shown in SEQ ID No. 1. The gRNA includes at least one gRNA unit and a termination sequence of 7 thymine bases. The gRNA units and the termination sequence of 7 thymine bases are ligated sequentially. The gRNA units are connected in series when the number of the gRNA units is greater than or equal to 2. The gRNA unit include sequentially-connected mature crRNA and a guide sequence of target gene.

Optionally, the P. patens of step 3) is P. patens of a protonema phase.

Optionally, the Cas12a-protease-encoding nucleotide sequence of step 1) is ligated to the plasmid pAct-Cas9 between a Ncol cleavage site and a Xbal cleavage site.

Optionally, based on 10 μL, the ligation system of step 1) includes 4.5 μL of the pAct-Cas9 plasmid, 3.5 μL of Cas12a-protease-encoding nucleotide sequence, 1 μL of T4 DNA ligase, and 1 μL of T4 DNA ligation buffer. The ligation is a ligation at 4° C. for 9-12 h.

Optionally, the gRNA of step 2) is ligated to the plasmid pU6-sgRNA between the Ncol cleavage site and the Xbal cleavage site.

Optionally, based on 10 μL, the ligation system of step 2) includes 3 μL of the pU6-sgRNA plasmid, 5 μL of gRNA, 1 μL of T4 DNA ligase, and 1 μL of T4 DNA ligation buffer. The ligation is a ligation at 4° C. for 9-12 h.

Optionally, in step 3) the volume ratio of the Cas12a protease expression vector, the gRNA expression vector and the plasmid for screening of resistance expression is 0.5-1.5:0.5-1.5:0.5-1.5. The concentration of the Cas12a protease expression vector is 0.5-1.5 μg/L. The concentration of the gRNA expression vector is 0.5-1.5 μg/μL. The concentration of the plasmid for screening of resistance expression is 0.5-1.5 μg/L.

Optionally, the plasmid for screening of resistance expression of step 3) is a plasmid for screening of hygromycin-resistance expression.

Optionally, the nucleotide sequence of the mature crRNA is shown in SEQ ID No. 2.

### BRIEF DESCRIPTION OF THE DRAWINGS

FIG. 1 represents a schematic structure view of a Cas12a protease expression vector and a gRNA expression vector.

FIG. 2 shows a backbone diagram of the Cas12a protease expression vector in Embodiment 1.

FIG. 3 shows a backbone diagram of the gRNA protease expression vector in Embodiment 1.

FIG. 4 shows the conditions of conducting gene editing of a transcription factor 9250 in P. patens using the CRISPR-Cas12a gene editing system.

WT, 9250-25, 9250-26, 9250-27, 9250-28, 9250-30, 9250-31 is shown in SEQ ID No. 19;

9250-2 is shown in SEQ ID No. 20.

9250-3 is shown in SEQ ID No. 21.

9250-4 is shown in SEQ ID No. 22.

9250-5 is shown in SEQ ID No. 23.

9250-6 is shown in SEQ ID No. 24.

9250-7 is shown in SEQ ID No. 25.

9250-8 is shown in SEQ ID No. 26.

9250-9 is shown in SEQ ID No. 27.

9250-10 is shown in SEQ ID No. 28.

9250-11 is shown in SEQ ID No. 29.

9250-12 is shown in SEQ ID No. 30.

9250-13 is shown in SEQ ID No. 31.

9250-14 is shown in SEQ ID No. 32.

9250-15 is shown in SEQ ID No. 33.

9250-17 is shown in SEQ ID No. 34.

9250-18 is shown in SEQ ID No. 35.

9250-19 is shown in SEQ ID No. 36.

9250-20 is shown in SEQ ID No. 37.

9250-21 is shown in SEQ ID No. 38.

9250-22 is shown in SEQ ID No. 39.

9250-23 is shown in SEQ ID No. 40.

9250-24 is shown in SEQ ID No. 41.

9250-29 is shown in SEQ ID No. 42.

9250-32 is shown in SEQ ID No. 43.

9250-33 is shown in SEQ ID No. 44.

FIG. 5 shows the conditions of conducting gene editing of a transcription factor 32480 in P. patens using the CRISPR-Cas12a gene editing system.

WT, 32480-8, 32480-19, 32480-20 and 32480-23 is shown in SEQ ID No. 45.

32480-2 is shown in SEQ ID No. 46.

32480-3 is shown in SEQ ID No. 47.

32480-4 is shown in SEQ ID No. 48.

32480-5 is shown in SEQ ID No. 49.

32480-6 is shown in SEQ ID No. 50.

32480-7 is shown in SEQ ID No. 51.

32480-9 is shown in SEQ ID No. 52.

32480-10 is shown in SEQ ID No. 53.

32480-11 is shown in SEQ ID No. 54.

32480-12 is shown in SEQ ID No. 55.

32480-13 is shown in SEQ ID No. 56.

32480-14 is shown in SEQ ID No. 57.

32480-15 is shown in SEQ ID No. 58.

32480-17 is shown in SEQ ID No. 59.

32480-18 is shown in SEQ ID No. 60.

32480-21 is shown in SEQ ID No. 61.

32480-22 is shown in SEQ ID No. 62.

32480-24 is shown in SEQ ID No. 63.

32480-25 is shown in SEQ ID No. 64.

32480-26 is shown in SEQ ID No. 65.

32480-27 is shown in SEQ ID No. 66.

32480-28 is shown in SEQ ID No. 67.

32480-29 is shown in SEQ ID No. 68.

32480-30 is shown in SEQ ID No. 69.

32480-31 is shown in SEQ ID No. 70.

32480-32 is shown in SEQ ID No. 71.

32480-33 is shown in SEQ ID No. 72.

FIG. 6 shows the conditions of conducting gene editing of a transcription factor 9580 in P. patens using the CRISPR-Cas12a gene editing system.

WT, 9580-2, 9580-6, 9580-10, 9580-11, 9580-12, 9580-17, 9580-18, 9580-19, 9580-21, 9580-22, 9580-24, 9580-30, 9580-31, 9580-32 and 9580-33 is shown in SEQ ID No. 73.

9580-3 is shown in SEQ ID No. 74.

9580-4 is shown in SEQ ID No. 75.

9580-5 is shown in SEQ ID No. 76.

9580-7 is shown in SEQ ID No. 77.

9580-8 is shown in SEQ ID No. 78.

9580-9 is shown in SEQ ID No. 79.

9580-13 is shown in SEQ ID No. 80.

9580-14 is shown in SEQ ID No. 81.

9580-15 is shown in SEQ ID No. 82.

9580-20 is shown in SEQ ID No. 83.

9580-23 is shown in SEQ ID No. 84.

9580-25 is shown in SEQ ID No. 85.

9580-26 is shown in SEQ ID No. 86.

9580-27 is shown in SEQ ID No. 87.

9580-28 is shown in SEQ ID No. 88.

9580-29 is shown in SEQ ID No. 89.

9580 in 9580-30 is shown in SEQ ID No. 90.

FIG. 7 shows the off-targeting sites which possibly occurs when gene editing of the transcription factor 9250 in P. patens is conducted by using the gRNA designed with the CRISPR-Cas12a gene editing system.

9250-ot-2, 9250-ot-4, 9250-ot-8, 9250-ot-9, 9250-ot-11, 9250-ot-19, 9250-ot-23, 9250-ot-28, 9250-ot-30 and 9250-ot-32 is shown in SEQ ID No. 91.

9250-ot-6 is shown in SEQ ID No. 92.

9250-ot-7 is shown in SEQ ID No. 93.

FIG. 8 shows the off-targeting sites which possibly occurs when gene editing of the transcription factor 32480 in P. patens is conducted by using the gRNA designed with the CRISPR-Cas12a gene editing system.

WT, 32480ot-2, 32480ot-7, 32480ot-8, 32480ot-9, 32480ot-10, 32480ot-20, 32480ot-21, 32480ot-23, 32480ot-22, 32480ot-27, 32480ot-28 and 32480ot-29 is shown in SEQ ID No. 94.

32480ot-3 is shown in SEQ ID No. 95.

FIG. 9 shows the off-targeting sites which possibly occur when gene editing of the transcription factor 9580 in P. patens is conducted by using the gRNA designed with the CRISPR-Cas12a gene editing system. 9580-ot-2, 9580-ot-3, 9580-ot-4, 9580-ot-5, 9580-ot-6, 9580-ot-7, 9580-ot-8, 9580-ot-9, 9580-ot-10/9580-ot-11, 9580-ot-17, 9580-ot-18, 9580-ot-19, 9580-ot-20, 9580-ot-21, 9580-ot-24, 9580-ot-25, 9580-ot-26, 9580-ot-27, 9580-ot-28 is shown in SEQ ID No. 96.

### DETAILED DESCRIPTION

In some embodiments, the present disclosure provides the application of a CRISPR/Cas12a gene editing system in the gene editing of P. patens, including the following steps.

1) Ligating a Cas12a-protease-encoding nucleotide sequence with nuclear localization signals at both ends to a plasmid pAct-Cas9, and initiating expression by a pActin promoter to obtain a Cas12a protease expression vector.

2) Ligating a gRNA to a plasmid pU6-sgRNA, and initiating expression by a PpU6 promoter to obtain a gRNA expression vector.

3) Transforming P. patens by using the Cas12a protease expression vector of step 1), the gRNA expression vector of step 2), and a plasmid for screening of resistance expression to obtain a mutant plant through screening for resistance.

There is no limitation on the temporal order of step 1) and step 2).

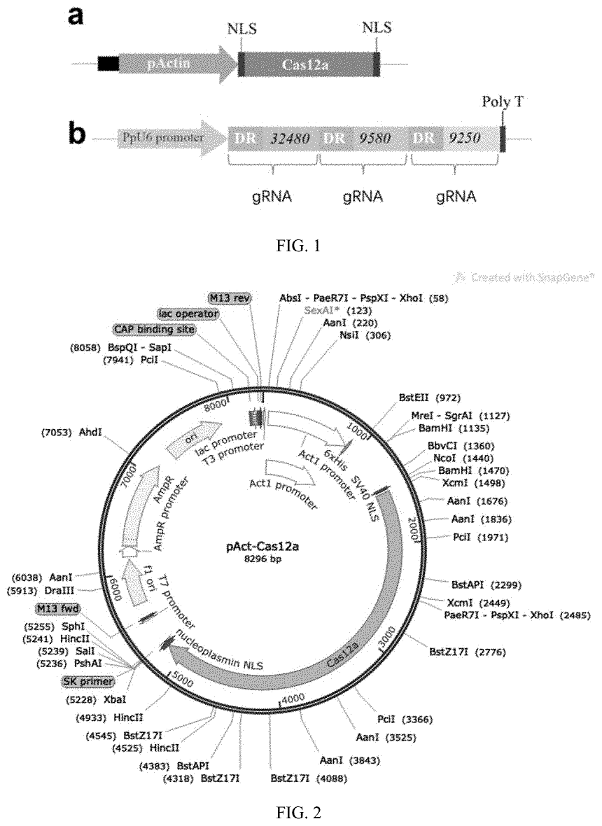

In other embodiments, the schematic structure view of the Cas12a protease expression vector and the gRNA expression vector is shown in FIG. 1. And in FIG. 1, a represents the Cas12a protease expression vector, and b represents the gRNA expression vector.

In further embodiments, the principle is that, a gRNA and a Cas12a protease form a complex, the gRNA directs the Cas12a protease to reach a target sequence containing the sequence of 5′-TTTN-3′PAM, complementary base pairing occurs between the gRNA and a DNA target sequence, the Cas12a protease conducts cleave downstream of the PAM sequence to break the double strand and thus produce a sticky end. Repairing is then conducted in a manner of homologous recombination or non-homologous end joining. Conditions of editing such as base insertion, deletion, or substitution occur during the repair process, thereby achieving the purpose of gene knockout.

According to an embodiment of the disclosure, a Cas12a-protease-encoding nucleotide sequence with nuclear localization signals at both ends is ligated to a plasmid pAct-Cas9, and expression is firstly initiated by a pActin promoter to obtain the Cas12a protease expression vector. The pActin promoter is a promoter left from the original vector pActCas9 and is located at the 5′ terminus of the Cas12a-protease-encoding nucleotide sequence. The Cas12a-protease-encoding nucleotide sequence with nuclear localization signals at both ends is shown in SEQ ID No. 1. In an implementation of the disclosure, the Cas12a-protease-encoding nucleotide sequence with nuclear localization signals at both ends is synthesized by Shanghai Generay Biotech Co., Ltd.

According to another embodiment of the disclosure, the Cas12a-protease-encoding nucleotide sequence is optionally ligated to the plasmid pAct-Cas9 between a Ncol cleavage site and a Xbal cleavage site.

In an implementation of the disclosure, the Cas12a-protease-encoding nucleotide sequence with nuclear localization signals at both ends and the plasmid pAct-Cas9 are subjected to double enzyme digestion respectively, and then ligated after recovering.

In an implementation of the disclosure, based on 20 μL, the double enzyme digestion system for the Cas12a-protease-encoding nucleotide sequence with nuclear localization signals at both ends includes 10 μL (about 3 μg) of a Cas12a fragment carrying a nuclear localization signal, 2 μL of Ncol, 2 μL of Xbal, 2 μL of 10× CutSmart Buffer, and 4 μL of dd H2O. The double enzyme digestion procedure is at 37° C. for 4 h.

In an implementation of the disclosure, based on 30 μL, the double enzyme digestion system for the plasmid pAct-Cas9 includes 20 μL (about 3 μg) of the plasmid pAct-Cas9,2 μL of Ncol, 2 μL of Xbal, 3 μL of 10× CutSmart Buffer, and 3 μL of dd H2O. The double enzyme digestion procedure is at 37° C. for 4 h.

The disclosure has no specific limitation on the recovery method, and a DNA recovery method conventionally used in the art may be utilized. In an implementation of the disclosure, optionally an agarose gel DNA extraction kit is used for recovery. The agarose gel DNA extraction kit is optionally a SanPrep column DNA gel extraction kit available from Sangon Biotech (Shanghai) Co., Ltd. under a product code of B518131-0100.

In an implementation of the disclosure, based on 10 μL, the ligation system, in which the Cas12a-protease-encoding nucleotide sequence with nuclear localization signals at both ends is ligated to the plasmid pAct-Cas9, includes: 4.5 μL of the pAct-Cas9 plasmid, 3.5 μL of Cas12a-protease-encoding nucleotide sequence, 1 μL of T4 DNA ligase, and 1 μL of T4 DNA ligation buffer. The ligation is a ligation at 4° C. for 9-12 h.

According to a further embodiment of the disclosure, the gRNA is ligated to the plasmid pU6-sgRNA, and expression is initiated by the PpU6 promoter to obtain the gRNA expression vector. The PpU6 promoter is a promoter left from the original vector pU6-sgRNA, and is located at the 5′ terminus of the nucleotide sequence encoding a gRNA expression kit. The gRNA includes at least one gRNA unit and a termination sequence of 7 thymine bases. The gRNA units and the termination sequence of 7 thymine bases are ligated sequentially. The gRNA units are connected in series when the number of the gRNA units is greater than or equal to 2. The termination sequence of 7 thymine bases is then added. The gRNA unit include sequentially-connected mature crRNA and a guide sequence of target gene. The nucleotide sequence of the mature crRNA (DR) is shown in SEQ ID No. 2.

In an implementation of the disclosure, the guide sequence of target gene is optionally target genes 9250, 32480 and 9580. The guide sequences of target genes 9250, 32480 and 9580 are obtained by designing through a CRISPOR online software (http://crispor.tefor.net/)(Haeussler et al., 2016) and CRISPR-P 2.0 (http://crispr.hzau.edu.cn/CRISPR2/) (Liu et al., 2017). The nucleotide sequence of the 9250 is shown in SEQ ID No. 3. The nucleotide sequence of the 32480 is shown in SEQ ID No. 4. The nucleotide sequence of the 9580 is shown in SEQ ID No.5. The nucleotide sequence of the series-connected gRNA is shown in SEQ ID No. 6. The series-connected gRNA is synthesized by Shanghai Generay Biotech Co., Ltd.

In an implementation of the disclosure, the gRNA is optionally ligated to the plasmid pU6-sgRNA between the Ncol cleavage site and the Xbal cleavage site.

In an implementation of the disclosure, the gRNA and the plasmid pU6-sgRNA are respectively subjected to double enzyme digestion, and then ligated after recovering.

In an implementation of the disclosure, based on 20 μL, the double enzyme digestion system for the gRNA includes 13 μL (about 3 g) of a gRNA fragment, 2 μL of Ncol, 2 μL of Xbal, 2 μL of 10× CutSmart Buffer, and 1 μL of dd H2O. The double enzyme digestion procedure is at 37° C. for 2 h.

In an implementation of the disclosure, based on 30 μL, the double enzyme digestion system for the plasmid pU6-sgRNA includes 18 μL (about 3 μg) of the plasmid pU6-sgRNA, 2 μL of Ncol, 2 μL of Xbal, 2 μL of 10× CutSmart Buffer, and 5 μL of dd H2O. The double enzyme digestion procedure is at 37° C. for 4 h.

The disclosure has no specific limitation on the recovery method, and a DNA recovery method conventionally used in the art may be utilized. In an implementation of the disclosure, optionally a agarose gel DNA extraction kit is used for recovery. The agarose gel DNA extraction kit is optionally a SanPrep column DNA gel extraction kit available from Sangon Biotech (Shanghai) Co., Ltd. under a product code of B518131-0100.

In an implementation of the disclosure, based on 10 μL, the ligation system, in which the gRNA is ligated to the plasmid pU6-sgRNA, includes: 3 μL of the pU6-sgRNA plasmid, 5 μL of the gRNA, 1 μL of the T4 DNA ligase, and 1 μL of the T4 DNA ligation buffer. The ligation is a ligation at 4° C. for 9-12 h.

In an implementation of the disclosure, after the Cas12a protease expression vector and the gRNA expression vector are obtained, P. patens is transformed by using the Cas12a protease expression vector, the gRNA expression vector, and the plasmid for resistance expression to obtain a mutant plant through screening for resistance.

The disclosure has no specific limitation on the transformation method, and a plasmid transformation method conventionally used in the art may be used. In an implementation of the disclosure, the transformation method is optionally a PEG-mediated protoplast method. The disclosure has no specific limitation on the screening method for resistance, and a screening method for resistance conventionally used in the art may be used.

In some embodiments of the disclosure, P. patens optionally are P. patens of protonema stage and gametophyte stage. The P. patens of the protonema stage and the gametophyte stage are optionally prepared by using the method including following steps. 1) P. patens is cultured by using a BCDAT medium for 5 days, to obtain a protonema-phase material for transformation. 2) The protonema-phase material of step 1) is cultured to obtain the P. patens of the gametophyte stage.

In the disclosure, firstly P. patens is cultured by using a BCDAT medium to obtain a protonema. The photoperiod of the culture is optionally 16 h light/8 h dark. The culture temperature is optionally 25° C. The illumination intensity of the culture is optionally 80 μmol photons m⁻²·s⁻¹. The culture time is 5 days.

In the disclosure, the formulation of the BCDAT medium is: 1 μM of MgSo4.7H2O, 18.4 μM of KH2PO4, 10 μM of KNO3, 45 μM of FeSO4.7H2O, 0.22 μM of CuSO4.5H2O, 10 μM of H3B03, 0.23 μM of CoCl2.6H2O, 0.1 μM of Na2MoO4.2H2O, 0.19 μM of ZnS04.7H2O, 2 μM of MnCl2.4H2O, 0.17 μM of KI, 5 mM of ammonium tartrate, and 0.8% of agar. Sterilization is conducted at 121° C. for 20 min.

In the disclosure, after obtaining of the protonema, the protonema cultured for one week is transferred onto and cultured on a BCDAT medium to obtain the P. patens of the gametophyte stage. The photoperiod of the culture is optionally 16 h light/8 h dark. The culture temperature is 25° C. The culture time is optionally 20-30 d, and more optionally 20 d. The illumination intensity of the culture is optionally 80 μmol photons m⁻²·s⁻¹.

In the disclosure, the plasmid for screening of resistance expression is optionally a plasmid for screening of hygromycin-resistance expression, and more optionally a plasmid BHRF for screening of hygromycin-resistance expression. The plasmid BHRF is kindly provided by the laboratory of professor Fabien Nogue at INRA Centre de Versailles-Grignon (referring to [Collonnier C, Epert A, Mara K, Maclot F, Guyon-Debast A, Chariot F, White C, Schaefer D G, Nogue F. 2016. CRISPR-Cas9-mediated efficient directed mutagenesis and RAD51-dependent and RAD51-independent gene targeting in the moss P. patens. Plant Biotechnology Journal]). The volume ratio of the Cas12a protease expression vector, the gRNA expression vector and the plasmid for screening of resistance expression is optionally 0.5-1.5:0.5-1.5:0.5-1.5, and more optionally 1:1:1. The concentration of the Cas12a protease expression vector is 0.5-1.5 μg/μL. The concentration of the gRNA expression vector is 0.5-1.5 μg/μL. The concentration of the plasmid for screening of resistance expression is 0.5-1.5 μL.

In the disclosure, after obtaining of the mutant plant, the target DNA sequence is amplified and then sequenced to obtain sequence information.

The disclosure has no specific limitation on the extraction of the whole genome DNA of the mutant plant, and a plant genome DNA extraction method conventionally used in the art may be used. In an implementation process of the disclosure, the whole genome DNA of the mutant plant is extracted by a CTAB method.

The disclosure has no specific limitation on the amplification method, and a DNA amplification method conventionally used in the art may be used. In an implementation the target DNA sequence is amplified using PCR amplification.

An exemplary application of the CRISPR/Cas12a gene editing system in the gene editing of P. patens as provided by the disclosure is described in detail in the following embodiment 1, but it should not be construed as limiting the claimed scope of the disclosure.

### Embodiment 1

Application of The CRISPR/Cas12a Gene Editing System in The Gene Editing of P patens

1. Cultivation Method of P. Patens

P. patens was cultured in a BCD AT medium. The protonema material cultured for 5 d was ground and sub-cultured, and then cultured under conditions of a photoperiod of 16 h light/8 h darkness, an illumination intensity of 80 umol m-2s-1, and 25° C. for 20 d to enter a uniform gametophyte stage.

2. Construction of Cas12a Protease Expression Vector

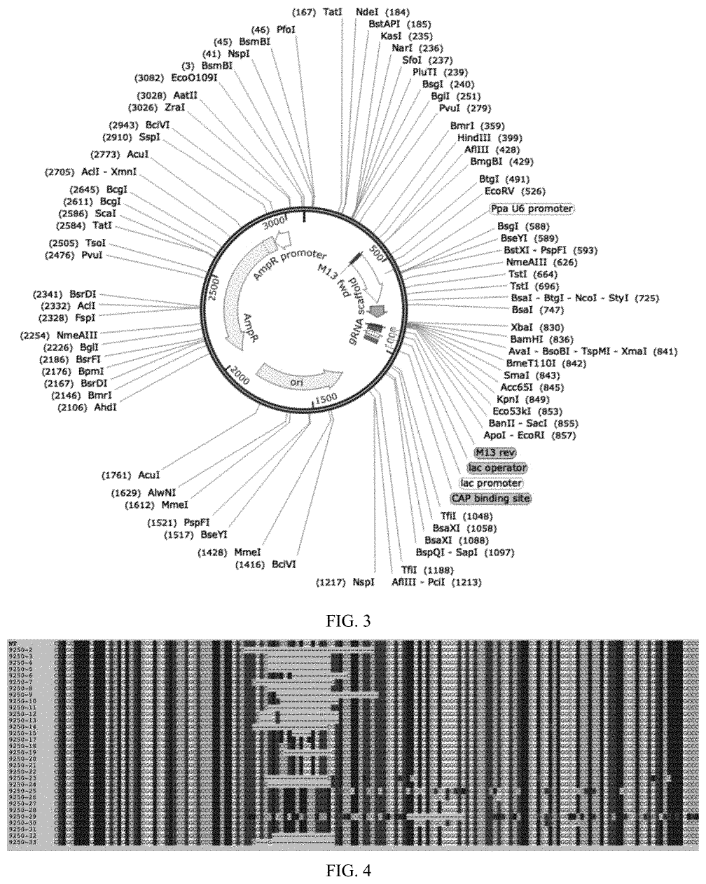

Nuclear localization signals (Nucleus Location Signal, NLS) were added to both ends of a nucleotide sequence of Cas12a. The Cas12a-protease-encoding nucleotide sequence with nuclear localization signals at both ends was synthesized by Shanghai Generay Biotech Co., Ltd. Then a Cas12a fragment carrying the nuclear localization signals was ligated to the plasmid pAct-Cas9 through a restriction enzyme ligation method at cleave sites Ncol and Xbal, and expression was initiated by using the pActinPpU6 promoter. The double enzyme digestion system for the Cas12a-protease-encoding nucleotide sequence with nuclear localization signals at both ends was shown in table 1. The double enzyme digestion system for the plasmid pAct-Cas9 was shown in table 2. The backbone diagram of the Cas12a protease expression vector was shown in FIG. 2.

TABLE 1
The double enzyme digestion system of the Cas12a-protease-encoding
nucleotide sequence with nuclear localization signals at both ends

| | | |
|---|---|---|
| The Cas12a-protease-encoding nucleotide | 10 | μL (about 3 μg) |
| sequence with nuclear localization signals at | | |
| both ends | | |
| NcoI | 2 | μL |
| Xba I | 2 | μL |
| 10x CutSmart Buffer | 2 | μL |
| dd H2O | 4 | μL |
| Total | 20 | μL |

Conditions and time of enzyme digestion: 37° C. for 4 h.

TABLE 2
The double enzyme digestion system for the plasmid pAct-Cas9

| | | |
|---|---|---|
| pAct-Cas9 vector | 20 | μL (about 3 μg) |
| NcoI | 2 | μL |
| Xba I | 2 | μL |
| 10x CutSmart Buffer | 3 | μL |
| dd H2O | 3 | μL |
| Total | 30 | μL |

Conditions and time of enzyme digestion: 37° C. for 4 h.

The aforementioned two enzyme digestion systems were identified by electrophoresis, and the target band was subjected to gel extraction (the agarose gel DNA extraction kit was purchased from Sangon Biotech (Shanghai) Co., Ltd. under the product name of SanPrep column DNA gel extraction kit used for recovery of PCR products, with the product code of B518131-0100).

The pAct-Cas9 vector fragment and the Cas12a target fragment were ligated with a T4 DNA ligase (available from Thermo Fisher Scientific under the product name of Thermo Scientific T4 DNA Ligase, with the product code of Ser. No. 00/532,665), and the ligation system in which the Cas12a-protease-encoding nucleotide sequence with nuclear localization signals at both ends was ligated to the plasmid pAct-Cas9 was shown in table 3.

TABLE 3
The ligation system in which the Cas12a-protease-encoding nucleotide
sequence with nuclear localization signals at both ends was ligated to
the plasmid pAct-Cas9

| | | |
|---|---|---|
| pAct-Cas9 vector fragment | 4.5 | μL |
| Cas12a target fragment | 3.5 | μL |
| T4 DNA ligase | 1 | μL |
| T4 DNA ligation Buffer | 1 | μL |
| Total | 10 | μL |

Conditions and time for ligation: 4° C. for 12 h.

3. Construction of gRNA Expression Vector

A gRNA sequence, in which three target genes (9250, 32480, and 9580) were knocked out simultaneously, was designed. Each gRNA consisted of two parts: a DR (direct repeat, i.e. mature crRNA) and a guide sequence of target gene, and 3 gRNA sequences were ligated together and synthesized by Shanghai Generay Biotech Co., Ltd. The gRNA fragment, in which three target sequences were knocked out, was ligated to the plasmid pU6-sgRNA by a restriction enzyme ligation method at the cleavage sites of Ncol and Xbal, and expression was initiated by the PpU6 promoter. The double enzyme digestion system for the gRNA was shown in Table 4. The double enzyme digestion system for the plasmid pU6-sgRNA was shown in Table 5.

TABLE 4
Double enzyme digestion system for gRNA

| | | |
|---|---|---|
| 3 gRNA fragments | 13 | μL (about 3 μg) |
| NcoI | 2 | μL |
| Xba I | 2 | μL |
| 10x CutSmart Buffer | 2 | μL |
| dd H2O | 1 | μL |
| Total | 20 | μL |

Conditions and time of enzyme digestion: 37° C. for 2 h.

TABLE 5
The double enzyme digestion system for the plasmid pU6-sgRNA

| | | |
|---|---|---|
| pU6-sgRNA vector | 18 | μL (about 3 μg) |
| NcoI | 2 | μL |
| Xba I | 2 | μL |
| 10x CutSmart Buffer | 3 | μL |
| dd H2O | 5 | μL |
| Total | 30 | μL |

Conditions and time of enzyme digestion: 37° C. for 4 h.

The aforementioned two enzyme digestion systems were identified by electrophoresis, and the target band was subjected to gel extraction (the agarose gel DNA extraction kit was purchased from Sangon Biotech (Shanghai) Co., Ltd. under the product name of SanPrep column DNA gel extraction kit used for recovery of PCR products, with the product code of B518131-0100).

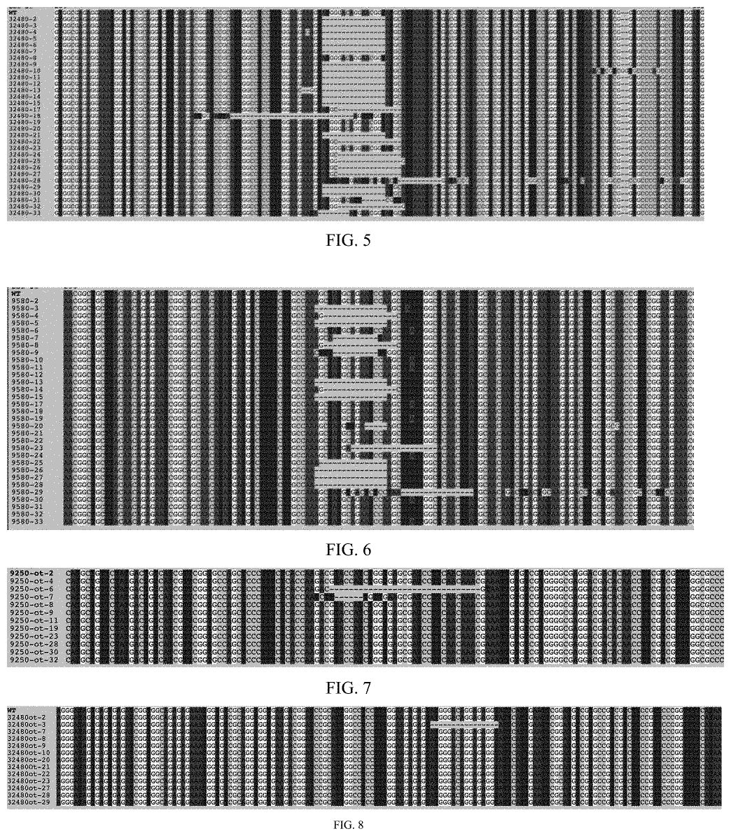

The pU6-sgRNA plasmid fragment and the 3 gRNA target fragments were ligated with the T4 DNA ligase (available from Thermo Fisher Scientific under the product name of Thermo Scientific T4 DNA Ligase with the product code of Ser. No. 00/532,665). The ligation system in which the gRNA was ligated to the plasmid pU6-sgRNA was shown in Table 6. The backbone diagram of the gRNA expression vector was shown in FIG. 3

TABLE 6
The ligation system in which the gRNA was ligated to the plasmid
pU6-sgRNA

| | | |
|---|---|---|
| pU6-sgRNA vector fragment | 3 | μL |
| 3 gRNA target fragments | 5 | μL |
| T4 DNA ligase | 1 | μL |
| T4 DNA ligation Buffer | 1 | μL |
| Total | 10 | μL |

Conditions and time of enzyme digestion: 4° C. for 12 h.

4. Transformation of P. patens

The P. patens of the protonema material of step 1 was transformed. A method of introducing an exogenous plasmid DNA into a protoplast as mediated by polyethylene glycol (PEG) was used. Here, the plasmid BHRF for screening of resistance expression (hygromycin resistance, at a concentration of 1 μg/μL, 10 μL), the Cas12a expression vector (at a concentration of 1 μg/μL, 10 μL), and the gRNA expression vector (at a concentration of 1 μg/μL, 10 μL) were mixed together. The P. patens was then transformed with a PEG-mediated protoplast method. The steps are as follows.

1) A driselase was prepared (the driselase was used for lysing the P. patens, breaking the cell wall, and releasing the protoplast, and was provided by the Hasabe laboratory of Japan): 0.5 g driselase+25 mL 8% mannitol were weighed, then shaken in a shaker with protection from light at 28° C. for 30 min, centrifuged at 5000 rpm for 10 min, and then subjected to filter sterilization through a 0.45 m filter head.

2) The P. patens material was added into the filtered driselase for lysing, then placed in a manual climatic box with protection from light for 30 minutes, and gently shaken every 10 minutes during the 30 minutes, then observed under a microscope for protoplast lysis, and if the majority of the protoplasts were lysed, the next step could be performed.

3) Formulation of a solution for dissolving PEG: 1 mL 1M Ca(No₃)₂+100 μL 1M Tris-HCl (pH 8.0)+9 mL 8% mannitol were subjected to filter sterilization with a 0.22 m 17 filter head, and then 5 mL was taken and added into 2 g PEG4000 to dissolve the PEG4000 under heat.

4) Formulation of a 3M solution: 0.91 g mannitol solid+0.15 mL MgCl (1M)+1 mL 1% MES (pH 5.6)+8.85 mL H2O were subjected to filter sterilization with a 0.22 m filter head.

5) The funnel filter paper was washed with 8% mannitol, then the lysed protoplasts were filtered into a 50 mL centrifuge tube, and then washed with 8% mannitol to a volume of 30 mL.

6) Centrifuging was conducted at 1200 rpm for 8 min.

7) The supernatant was pipetted with a pipette (it should be careful not to pipette the whole supernatant, some liquid was left, and then the remaining solution was mixed gently in the palm).

8) Then the mixture was washed with 30 mL of 8% mannitol (washing for the first pass).

9) Centrifuging was conducted at 1200 rpm for 8 min.

10) The supernatant was pipetted with a pipette (it should be careful not to pipette the whole supernatant, some liquid was left, and then the remaining solution was mixed gently in the palm).

11) Then the mixture was washed with 30 mL of 8% mannitol (washing for the second pass).

12) Centrifuging was conducted at 1200 rpm for 8 min.

13) The supernatant was pipetted with a pipette (it should be careful not to pipette the whole supernatant, some liquid was left, and then the remaining solution was mixed gently in the palm).

14) 40 mL of 8% mannitol was added, and then microscopic examination was conducted to perform protoplast counting, and 100 μL of liquid was drawn and then counted with a blood counting chamber under a microscope.

15) Centrifuging was conducted at 1200 rpm for 8 min, the supernatant was pipetted with a pipette, then added with (the number of protoplasts*1,000)/4 μL of the 3M solution, so as to obtain protoplast solution.

16) 30 μg (about 30 μL) of the plasmid was added to a 15 mL heat shock tube, then added with 300 μL of protoplast solution, and finally added with 300 μL of PEG solution, and shaken immediately.

17) The mixture was subjected to heat shock at 45° C. for 5 min, and then placed in cool water for 10 min.

18) 300 μL, 600 μL, 1 mL, 3 mL of 8% mannitol were added sequentially, and shaken immediately after each addition.

19) Centrifuging was conducted at 1200 rpm for 8 min, and then the supernatant was removed.

20) 1 mL CaCb was added into the formulated 40 mL Top argar, 10 mL of the mixture was taken and poured into the heat shock tube and shaken quickly, then poured onto a medium and placed in an illumination incubator for cultivation.

5. DNA Level Identification and Sequencing of Gene Editing Mutant

After the transformation of the P. patens protoplasts conducted by the PEG-mediated method was completed, 31 mutant plants were obtained through the following resistance screening. A process of resistance screening was: conducting microscopic observation at 3-5 days after the end of the transformation process to see if budding occurs. If most of the protoplasts were budded, then they were transferred onto a hygromycin-resistant medium for resistance screening, cultured on the resistant medium for about one week (the plants into which no resistant plasmid was transferred were died during the screening), then transferred to a normal medium for recovery growth, then transferred onto a hygromycin-resistant medium for the second-pass of resistance screening (hygromycin screening: the concentration for the first-pass screening was 20 μg/mL, and the concentration for the second-pass of screening was 40 μg/mL). About one week later, the protoplasts were transferred from the resistant medium to a normal medium for recovery growth. After the protoplasts were grown into individual plants, the seedlings were cultured separately. When the plants were grown up, DNA of them were extracted, and possible gene editing sites were subjected to PCR amplification and then sequenced to identify whether the plants had been edited.

In this experiment the total DNA extraction of the P. patens was conducted using the CTAB method, and the 2*Taq Master Mix of nonoprotein company was used to conduct PCR amplification of gene editing sites of three genes 9250, 32480 and 9580 respectively, and the amplified fragment had a size of about 500 bp.

System (50 μL): 1 μL of F (10 μM), 1 μL of R (10 μM), 25 μL of 2*Taq Master Mix, 22 μL of H2O, and 1 μL of genomic DNA.

An eppendorf PCR instrument was used, and the procedure was: pre-denaturation at 95° C. for 5 min, denaturation at 95° C. for 30 s, annealing at 53° C. for 15 s, 20 extension at 72° C. for 30 s (with the amplification efficiency of 1 kb/min), 34 cycles, then extension at 72° C. for 10 min after the cycles, and incubation at 4° C. The PCR stock solutioncontaining the size bands of target fragments was sent for sequencing, to observe the gene editing conditions of the three genes 9250, 32480 and 9580. The upstream primer for PCR amplification of 9580 was as shown in SEQ ID No. 7. The downstream primer for PCR amplification of 9580 was as shown in SEQ ID No. 8. The upstream primer for PCR amplification of 32480 was as shown in SEQ ID No. 9. The downstream primer for PCR amplification of 32480 was as shown in SEQ ID No. 10. The upstream primer for PCR amplification of 9250 was as shown in SEQ ID No. 11. The downstream primer for PCR amplification of 9250 was as shown in SEQ ID No. 12.See Table 7 for details.

TABLE 7
Primers used in DNA amplification of target sequences

| target | Upstream Primer | Downstream Primer | length of PCR |
|---|---|---|---|
| gene | (5′ to 3′) | (5′ to 3′) | product (bp) |
| Pp_9580 | CTGTATATGTGTTAACGAAACG | GACGCCAGATTGTCGATTCAGT | 580 bp |
| | (SEQ ID No. 7) | (SEQ ID No. 8) | |
| Pp_32480 | GAGTTCTTAGTCGTGCTTCGCG | GCTGGAAAAGTTGTTGTGCTTA | 567 bp |
| | (SEQ ID No. 9) | (SEQ ID No. 10) | |
| Pp_9250 | CGGACCTGTAAGCTAGTCCTT | TGTATTACTCATTTGGACGGC | 500 bp |
| | (SEQ ID No. 11) | (SEQ ID No. 12) | |

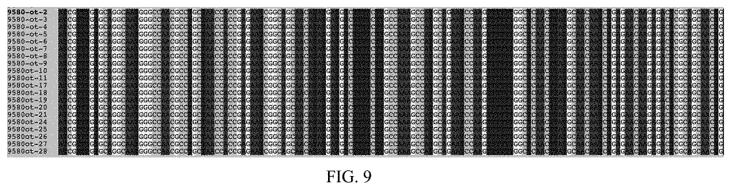

Identification results: see FIGS. 4-6 and Table 8 for the gene editing conditions of the P. patens. FIG. 4 showed the conditions of conducting gene editing of a transcription factor 9250 in P. patens by using the CRISPR-Cas12a gene editing system. FIG. 5 showed the Conditions of conducting gene editing of a transcription factor 32480 in P. patens by using the CRISPR-Cas12a gene editing system. FIG. 6 showed the conditions of conducting gene editing of a transcription factor 9580 in P. patens by using the CRISPR-Cas12a gene editing system.

FIG. 4 represented that 27 ones of the 31 plants had the editing of 9250 at an editing efficiency of 87.1%. FIG. 5 represented that 27 ones of the 31 plants had the editing of 32480 at an editing efficiency of 87.1%. FIG. 6 represented 16 ones of the 31 plants had the editing of 9580 at an editing efficiency of 51.6%. Table 8 represented that the statistical editing efficiencies of the triple-gene mutants, double-gene mutants and single-gene mutants were 38.7%), 45.2%), and 16.1% respectively. It could be seen from this that, the CRISPR-Cas12a gene editing system was very efficient in multi-gene editing of P. patens.

TABLE 8
Editing efficiencies of triple-gene mutants, double-gene mutants and

| Type | Groups | | | |
|---|---|---|---|---|
| Number of mutated genes | 3 | 2 | 1 | 0 |
| Number of transgenic plants | 12 | 14 | 5 | 0 |
| Editing Efficiency | 38.7% | 45.2% | 16.1% | 0 |

6. Detection of Off-Target Sites

In order to detect whether an off-target effect occurs in the CRISPR-Cas12a gene editing system, we conducted the detection according to the potential off-target sites predicted on the website (http:.//crispor.tefor.net). The fragments of the off-target sites were subjected to PCR amplification, and the amplified fragment had a size of about 500 bp.22 System (50 μL): 1 μL of F (10 μM), 1 μL of R (10 μM), 25 μL of 2*Taq Master Mix, 22 μL of H2O, and 1 μL of genomic DNA. The eppendorf PCR instrument was used, and the procedure was: predenaturation at 95° C. for 5 min, denaturation at 95° C. for 30 s, annealing at 53° C. for 15 s, extension at 72° C. for 30 s (with the amplification efficiency of 1 kb/min), 34 cycles, then extension at 72° C. for 10 min after the cycles, and incubation at 4° C.

The PCR stock solution containing the size bands of target fragments was sent for sequencing, to observe whether the DNA sequences of the off-target sites that might be caused by the gRNAs designed through gene editing of the three genes 9250, 32480, and 9580 were edited. If the editing occurs, it demonstrated that there was the off-target problem, and if not, it demonstrated that there was no off-target problem. The upstream primer for PCR amplification of 9580 was as shown in SEQ ID No. 13. The downstream primer for PCR amplification of 9580 was as shown in SEQ ID No. 14. The upstream primer for PCR amplification of 32480 was as shown in SEQ ID No. 15. The downstream primer for PCR amplification of 32480 was as shown in SEQ ID No. 16. The upstream primer for PCR amplification of 9250 was as shown in SEQ ID No. 17. The downstream primer for PCR amplification of 9250 was as shown in SEQ ID No. 18. See Table 9 for details.

TABLE 9
Primers used in DNA amplification of off-target sites

| target | Upstream Primer | Downstream Primer | length of PCR |
|---|---|---|---|
| gene | (5′ to 3′) | (5′ to 3′) | product (bp) |
| Pp_9580 | GACCATATGGCTTTTGATGAA | TCGCGAGTGTACCTACGTCT | 514 bp |
| | (SEQ ID No. 13) | (SEQ ID No. 14) | |
| Pp_32480 | TCGCAGGTGGTGAAGACGGAT | TTCAGCCGCGTCAAGATTGAA | 470 bp |
| | (SEQ ID No. 15) | (SEQ ID No. 16) | |
| Pp_9250 | TTTGGCTCTGTACGTAGATTG | CACTTCTCACTGAAACGCTAC | 466 bp |
| | (SEQ ID No. 17) | (SEQ ID No. 18) | |

Detection Results: see FIGS. 7-9 for the schematic views of the detection results of the predicted potential off-target sites. FIG. 7 showed the off-targeting sites which possibly occurred when gene editing of the transcription factor 9250 in P. patens was conducted by using the gRNA designed with the CRISPR-Cas12a gene editing system. FIG. 8 showed the off-targeting sites which possibly occurred when gene editing of the transcription factor 32480 in P. patens was conducted by using the gRNA designed with the CRISPR-Cas12a gene editing system. FIG. 9 showed the off-targeting sites which possibly occurred when gene editing of the transcription factor 9580 in P. patens was conducted by using the gRNA designed with the CRISPR-Cas12a gene editing system. It could be found that, very little off-target occurred when the designed gRNA edited 9250 and 32,480. Only 2 plants of randomly selected 12 plants having editing of 9250 had the occurrence of off-target, with an off-target ratio of 16.6%. Only 1 plants of randomly selected 13 plants having editing of 32480 had the occurrence of off-target, with an off-target ratio of 0.07%. No plant of randomly selected 20 plants having editing of 9580 had the occurrence of off-target, with an off-target ratio of 0. Therefore, the probability of off-targeting during multi-gene editing conducted by the CRISPR-Cas12a is very small.

Various embodiments of the disclosure may have one or more of the following effects. The disclosure may provide the application of a CRISPR/Cas12a gene editing system in the gene editing of P. patens. The application may have a high gene editing efficiency and/or a low off-target probability. The application may be capable of conducting editing of multiple targets simultaneously with high efficiency. By constructing a Cas12a protease expression vector and a gRNA expression vector, a gRNA and a Cas12a protease form a complex, the gRNA directs the Cas12a protease to reach the vicinity of a target sequence containing the sequence of 5′-TTTN-3′PAM, complementary base pairing occurs between the gRNA and a DNA target sequence, the Cas12a protease conducts cleave downstream of the PAM sequence to break the double strand and thus produce a sticky end. Repairing may be then conducted in a manner of homologous recombination or non-homologous end joining. Conditions of editing such as base insertion, deletion or, substitution occur during the repair process, thereby achieving the purpose of gene knockout. The CRISPR/Cas12a gene editing system of the disclosure may have relatively higher multi-gene editing efficiency when applied in gene editing of P. patens. The three transcription factors of the P. patens may be edited by the CRISPR/Cas12a gene editing system, and the editing efficiencies of the corresponding triple-gene mutant, double-gene mutant and single-gene mutant may be respectively 38.7%, 45.2% and 16.1%. The probability of off-targeting during multi-gene editing conducted by the CRISPR-Cas12a may be very small.

The foregoing descriptions are only optional implementation manners of the present invention. It should be noted that for a person of ordinary skill in the art, several improvements and modifications may further be made without departing from the principle of the present invention. These improvements and modifications should also be deemed as falling within the protection scope of the present invention.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1. A method for editing a gene of Physcomitrella patens (P. patens) with a CRISPR/Cas12a gene editing system, comprising the steps of:
1) ligating a Cas12a-protease-encoding nucleotide sequence with nuclear localization signals at both ends to a plasmid pAct-Cas9, and initiating expression by a pActin promoter to obtain a Cas12a protease expression vector;
2) ligating a gRNA to a plasmid pU6-sgRNA, and initiating expression by a PpU6 promoter to obtain a gRNA expression vector; and
3) transforming P. patens by using the Cas12a protease expression vector of step 1), the gRNA expression vector of step 2), and a plasmid for screening of resistance expression to obtain a mutant plant through screening for resistance;
wherein:
there is no limitation on a temporal order of step 1) and step 2);
the Cas12a-protease-encoding nucleotide sequence with nuclear localization signals at both ends is shown in SEQ ID No. 1;
the gRNA comprises at least one gRNA unit and a termination sequence of 7 thymine bases;
the gRNA units and the termination sequence of 7 thymine bases are ligated sequentially;
the gRNA units are connected in series when a number of the gRNA units is greater than or equal to 2; and
the gRNA units comprise sequentially-connected mature crRNAs and a guide sequence of target gene.

2. The method according to claim 1, wherein the P. patens of step 3) is P. patens of a protonema phase.

3. The method according to claim 2, wherein the Cas12a-protease-encoding nucleotide sequence of step 1) is ligated to the plasmid pAct-Cas9 between a Ncol cleavage site and a Xbal cleavage site.

4. The method according to claim 3, wherein:
a ligation system of step 1) has a volume of 10 μL;
the ligation system of step 1) comprises 4.5 μL of the pAct-Cas9 plasmid, 3.5 μL of Cas12a-protease-encoding nucleotide sequence, 1 μL of T4 DNA ligase, and 1 μL of T4 DNA ligation buffer; and
the ligation is a ligation at 4° C. for 9-12 h.

5. The method according to claim 2, wherein the gRNA of step 2) is ligated to the plasmid pU6-sgRNA between a Ncol cleavage site and a Xbal cleavage site.

6. The method according to claim 5, wherein:
a ligation system of step 2) has a volume of 10 μL;
the ligation system of step 2) comprises 3 μL of the pU6-sgRNA plasmid, 5 μL of gRNA, 1 μL of T4 DNA ligase, and 1 μL of T4 DNA ligation buffer; and
the ligation is a ligation at 4° C. for 9-12 h.

7. The method according to claim 2, wherein:
a volume ratio of the Cas12a protease expression vector, the gRNA expression vector and the plasmid for screening of resistance expression is 0.5-1.5:0.5-1.5:0.5-1.5 in step 3);
a concentration of the Cas12a protease expression vector is 0.5-1.5 μg/μL;
a concentration of the gRNA expression vector is 0.5-1.5 μg/μL; and
a concentration of the plasmid for screening of resistance expression is 0.5-1.5 μg/μL.

8. The method according to claim 1, wherein the Cas12a-protease-encoding nucleotide sequence of step 1) is ligated to the plasmid pAct-Cas9 between a Ncol cleavage site and a Xbal cleavage site.

9. The method according to claim 8, wherein:
a ligation system of step 1) has a volume of 10 μL;
the ligation system of step 1) comprises 4.5 μL of the pAct-Cas9 plasmid, 3.5 μL of Cas12a-protease-encoding nucleotide sequence, 1 μL of T4 DNA ligase, and 1 μL of T4 DNA ligation buffer; and
the ligation is a ligation at 4° C. for 9-12 h.

10. The method according to claim 1, wherein the gRNA of step 2) is ligated to the plasmid pU6-sgRNA between a Ncol cleavage site and a Xbal cleavage site.

11. The method according to claim 10, wherein:
a ligation system of step 2) has a volume of 10 μL;
the ligation system of step 2) comprises 3 μL of the pU6-sgRNA plasmid, 5 μL of gRNA, 1 μL of T4 DNA ligase, and 1 μL of T4 DNA ligation buffer; and
the ligation is a ligation at 4° C. for 9-12 h.

12. The method according to claim 1, wherein:
a volume ratio of the Cas12a protease expression vector, the gRNA expression vector and the plasmid for screening of resistance expression is 0.5-1.5:0.5-1.5:0.5-1.5 in step 3);
a concentration of the Cas12a protease expression vector is 0.5-1.5 μg/μL;
a concentration of the gRNA expression vector is 0.5-1.5 μg/μL; and
a concentration of the plasmid for screening of resistance expression is 0.5-1.5 μg/μL.

13. The method according to claim 1, wherein the plasmid for screening of resistance expression of step 3) is a plasmid for screening of hygromycin-resistance expression.

14. The method according to claim 1, wherein a nucleotide sequence of the mature crRNA is shown in SEQ ID No. 2.


SEQUENCE LISTING (18 sequences)

SEQ ID NO: 1 - DNA, 3788 bp, artificial sequence
  the Cas12a-protease-encoding nucleotide sequence with nuclear localization signals at both ends
catggccccaaagaagaagcggaaggtcgggatccacggagtcccagcagccatgagtaagctggagaaatttacgaattgttatagcctgtccaagacactgaggttcaaggctatccccgttggaaaaactcaagagaatatcgataacaagcgtctgctggttgaggatgagaagagagcggaagactataagggagtcaaaaaacttctggatagatactatctgtcttttataaacgacgtgctgcattccattaaattgaagaatctgaacaactatatttccctgttccgaaagaagacgagaacagagaaagaaaataaggagctggaaaacctggagatcaacctgaggaaggagattgcaaaggctttcaagggaaatgaaggttataagagcctgttcaaaaaagatattattgaaactatactgcctgaatttctcgatgataaggatgagatcgcgctcgtgaacagctttaacggattcacgactgcctttactggatttttcgataatagggagaacatgttctcagaggaagccaagagcacctcgatcgcttttcgttgcataaatgagaatttgacaagatacatcagcaacatggacattttcgagaaggttgatgcgatctttgataagcatgaggttcaggagattaaggagaagattctgaactctgattatgatgttgaggatttcttcgaaggagaattttttaatttcgttttgacacaggaaggtatagatgtgtataacgctattatcggtgggttcgttaccgaatcaggggagaagataaagggtctgaatgagtacatcaatctgtataaccagaagacaaagcagaaactgccaaaattcaagcctctctataagcaagtcctgagcgatcgggagtcgctttcgttctacggtgaaggttataccagcgatgaggaggtactggaggtctttagaaacactctgaacaagaatagcgaaattttctcttccattaagaagctggagaagctgttcaagaattttgatgagtactcgagcgcaggtattttcgtgaagaacggacctgctataagcactattagcaaggatatttttggagagtggaatgttattcgggataagtggaatgcagagtacgacgatatacacctgaagaagaaggctgtggtaactgagaagtatgaggacgatagacgcaaaagcttcaagaagatcggttcctttagcctggagcaactgcaggagtatgcggacgcagatctgagcgtggtcgagaaactgaaggagatcattatccaaaaggtggatgagatttacaaggtatacggtagctcagaaaagctctttgatgcagatttcgttctcgaaaagagcctgaagaagaatgatgctgttgttgctataatgaaggacctgctcgatagcgttaagagctttgagaattacatcaaggcattctttggcgagggaaaggaaacaaacagagacgaaagcttctatggcgactttgtgctagcttatgacatcctgctgaaggtagaccatatatatgatgcaattcgtaattacgttacccaaaagccgtacagcaaggataagttcaaactctattttcaaaacccgcaatttatgggtggctgggataaggacaaggaaacagattatagggccactatcctgcgatatggcagcaagtactacctcgctatcatggataagaagtatgctaagtgtctccagaagatagataaggacgacgtgaacggaaattacgagaagattaattacaagctactgcctgggcctaacaagatgctgccaaaagtcttcttttcaaagaaatggatggcttactacaacccatcagaggatatccaaaagatatacaaaaatgggacctttaaaaagggagacatgtttaacctgaatgactgccataagctgatcgatttttttaaggatagcatcagcagatatcctaagtggtcaaacgcttatgattttaatttctccgaaacagagaagtataaggacattgctggtttctatagggaagtcgaagaacaaggttataaggttagctttgaatcggcttcaaagaaggaagttgataaactagtggaagaaggtaagctctatatgttccaaatttacaacaaggatttttccgacaagtctcacggaactcctaaccttcatacgatgtattttaagctgctgtttgatgagaacaatcacggccaaatacgactctccggtggagcagaactttttatgcggagagcgagcctcaagaaagaagaacttgtggttcatcccgccaacagcccgattgctaacaagaatccagataaccctaagaagactacaacactgtcgtacgatgtttataaggataagcgtttctcggaagaccagtatgagttgcacataccaatagcaatcaataagtgcccaaagaacattttcaaaatcaacaccgaggttcgtgttctgctgaagcatgacgataacccgtacgtgataggaattgacaggggggagagaaacctgctgtatatcgtagtcgttgatggaaagggcaacatcgttgagcagtacagcttgaatgaaatcattaacaactttaacggtatacgtatcaagactgactaccatagcctgctggataagaaggagaaggagagattcgaggcaaggcaaaactggacgagcatcgaaaacatcaaggaactaaaggccggttatatcagccaagtagtccataagatttgtgagctggtggagaagtatgacgctgtcatcgccttggaggatctgaattctggcttcaaaaatagccgggtgaaagtggagaagcaggtataccaaaagtttgagaagatgctcatcgataagctaaactacatggttgataaaaagagcaatccgtgcgccacaggaggagctctcaaagggtaccaaataaccaacaagttcgaaagcttcaagtcaatgagcacacaaaacgggtttattttttatataccagcatggctgacgagcaagattgatcccagcactggttttgtcaacctgctgaagaccaagtatactagcattgctgattctaaaaagtttatttcgagcttcgaccgtattatgtacgttccagaggaggatctgttcgaattcgcgctggattacaagaacttcagcagaaccgatgctgattatattaagaagtggaagctgtactcttacggtaatagaataaggatcttcagaaaccctaagaaaaacaacgtgttcgattgggaagaggtctgtctcactagcgcgtataaggagctgttcaataagtacggaataaattaccagcaaggggacatccgcgctctgctgtgtgaacagagtgacaaagcattttatagcagtttcatggcgctgatgagcctgatgctgcagatgcgaaacagcattactggtagaacagacgttgactttctcatcagccctgtgaagaacagcgacggaatcttttacgattccaggaactacgaggcacaggaaaacgctattctgcctaaaaacgctgatgcaaacggagcctataacattgcacgaaaagttctgtgggctattggacagttcaagaaggctgaagatgagaagctagataaggtaaagatagctattagcaataaggaatggctggagtacgcacaaacatccgttaagcacaaaaggccggcggccacgaaaaaggccggccaggcaaaaaagaaaaagtaat

SEQ ID NO: 2 - DNA, 21 bp, artificial sequence
  the nucleotide sequence of the mature crRNA
taatttctactaagtgtagat

SEQ ID NO: 3 - DNA, 24 bp, artificial sequence
  the nucleotide sequence of the 9250
ttgaaggatcgttcaccagatggt

SEQ ID NO: 4 - DNA, 24 bp, artificial sequence
  the nucleotide sequence of the 32480
gaagaaagtatgggacaggaacgg

SEQ ID NO: 5 - DNA, 24 bp, artificial sequence
  the nucleotide sequence of the 9580
ttgccaaagctatgcagaatccaa

SEQ ID NO: 6 - DNA, 142 bp, artificial sequence
  the nucleotide sequence of the series-connected gRNA
taatttctactaagtgtagatgaagaaagtatgggacaggaacggtaatttctactaagtgtagatttgccaaagctatgcagaatccaataatttctactaagtgtagatttgaaggatcgttcaccagatggtttttttt

SEQ ID NO: 7 - DNA, 22 bp, artificial sequence
  The upstream primer for PCR amplification of 9580
ctgtatatgtgttaacgaaacg

SEQ ID NO: 8 - DNA, 22 bp, artificial sequence
  the downstream primer for PCR amplification of 9580
gacgccagattgtcgattcagt

SEQ ID NO: 9 - DNA, 22 bp, artificial sequence
  the upstream primer for PCR amplification of 32480
gagttcttagtcgtgcttcgcg

SEQ ID NO: 10 - DNA, 22 bp, artificial sequence
  the downstream primer for PCR amplification of 32480
gctggaaaagttgttgtgctta

SEQ ID NO: 11 - DNA, 21 bp, artificial sequence
  the upstream primer for PCR amplification of 9250
cggacctgtaagctagtcctt

SEQ ID NO: 12 - DNA, 21 bp, artificial sequence
  the downstream primer for PCR amplification of 9250
tgtattactcatttggacggc

SEQ ID NO: 13 - DNA, 21 bp, artificial sequence
  The upstream primer for PCR amplification of 9580 of off-target sites
gaccatatggcttttgatgaa

SEQ ID NO: 14 - DNA, 20 bp, artificial sequence
  the downstream primer for PCR amplification of 9580 of off-target sites
tcgcgagtgtacctacgtct

SEQ ID NO: 15 - DNA, 21 bp, artificial sequence
  the upstream primer for PCR amplification of 32480 of off-target sites
tcgcaggtggtgaagacggat

SEQ ID NO: 16 - DNA, 21 bp, artificial sequence
  the downstream primer for PCR amplification of 32480 of off-target sites
ttcagccgcgtcaagattgaa

SEQ ID NO: 17 - DNA, 21 bp, artificial sequence
  the upstream primer for PCR amplification of 9250 of off-target sites
tttggctctgtacgtagattg

SEQ ID NO: 18 - DNA, 21 bp, artificial sequence
  the downstream primer for PCR amplification of 9250 of off-target sites
cacttctcactgaaacgctac